(19) United States
(12) Patent Application Publication
(10) Pub. No.: US 2024/0299086 A1
(43) Pub. Date: Sep. 12, 2024

(54) INTRAUTERINE MORCELLATION DEVICE

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Charles A. Baker, Rogers, MN (US); Theodore C. Blus, Arden Hills, MN (US); Joey Magno, Dudley, MA (US); Jake Terravecchia, Brighton, MA (US); Jeffrey D. Holton, Stanchfield, MN (US)

(21) Appl. No.: 18/406,596

(22) Filed: Jan. 8, 2024

Related U.S. Application Data
(60) Provisional application No. 63/488,859, filed on Mar. 7, 2023.

(51) Int. Cl. A61B 18/14; A61B 17/00; A61B 18/00; A61B 90/00

(52) U.S. Cl. CPC A61B 18/1485; A61B 18/1492; A61B 2017/00296; A61B 2018/00559; A61B 2018/00601; A61B 2018/1435; A61B 2090/0811; A61B 2217/005; A61B 2218/007

(57) ABSTRACT

A medical device can include an elongate outer member and an elongate inner member in the elongate outer member. The elongate outer member defines a first lumen and includes a lateral suction port that is in fluid communication with the first lumen. The elongate inner member is rotatable relative to the elongate outer member and defines a second lumen. The second lumen includes an opening at a distal end of the elongate inner member to allow passage of fluid into the second lumen. The elongate inner member has a first lateral passage where the first lateral passage is rotatable into partial alignment with the lateral suction port to provide or control suction or other fluid communication between the lateral suction port and the second lumen. The medical device also has an electrode at a distal end of the inner member.

### PRIORITY CLAIM

This application claims the benefit of U.S. Provisional Patent Application No. 63/488,859, filed Mar. 7, 2023, the contents of which are claimed herein by reference in their entirety.

### TECHNICAL FIELD

This document pertains generally, but not by way of limitation, to surgical devices that can be used for various surgical procedures. More specifically, but not by way of limitation, the present application relates to a surgical device that may be used to treat the reproductive system of a female patient.

### BACKGROUND

Growths can occur in the lining of a uterus, which can cause discomfort and interfere with menstruation and fertility if left untreated. The growths can include polyps and fibroids. Uterine polyps are growths that can form in the inner lining of a uterus. Uterine polyps typically form when an overgrowth of endometrial tissue occurs. The polyp can attach to the endometrium and then extend into the uterus.

Fibroids are noncancerous growths that can develop in the wall of a uterus. Different types of fibroids include intramural, submucosal, subserosal, and pedunculated. Intramural fibroids can occur within the muscular walls of the uterus and cause heavy bleeding. Submucosal fibroids can occur inside the uterine cavity or can abut the uterine cavity. Submucosal fibroids can also cause heavy bleeding. Subserosal fibroids can occur on the outer wall of the uterus and can typically cause bulk or pressure symptoms. Pedunculated fibroids can attach to the uterine wall by a stalk-like growth called a peduncle.

When a patient has a submucosal fibroid, a hysteroscopic procedure can be used to examine the inside of the uterus and remove the fibroid via a hysteroscopic resection. A hysteroscope can be inserted through the vagina and through the natural opening of the cervix. Typically, due to the diameter of the hysteroscope, the cervix must be dilated, which can be painful and require the patient to be under general anesthesia. As a result, an operating room is required and the recovery period due to the dilation can be increased.

### SUMMARY

Accordingly, what is needed is a hysteroscope that is smaller in diameter that allows for less dilation of the cervix and less anesthesia. This problem can be addressed by providing a hysteroscope having an outer member that defines an outer lumen along a longitudinal axis of the outer member. An inner member can be partially disposed within the outer lumen. The outer member has a lateral suction port that can be in fluid communication with the outer lumen. The inner member, which can rotate within the outer member, can define an inner lumen along a longitudinal axis of the inner member.

The inner member can also have a first lateral passage that can rotate into alignment with the suction port. Thus, fluid and debris within the inner member can exit into the suction port via the lateral passage. Because the inner member is rotatable relative to the outer lumen, the inner member can rotate to adjust an alignment of the first lateral passage with the suction port such that the first lateral passage is at least partially aligned with the suction port. The degree of alignment can be used to establish or adjust an amount of suction passing through the first lateral passage. More particularly, the inner member can be rotated to adjust an amount of suction at a target site.

The inner member can have a second lateral passage located at a distal end of the inner member. The second lateral passage can provide an opening into the inner lumen of the inner member. The second lateral passage can extend between the opening at the distal end of the inner lumen and the closed end of the inner lumen. The interior cavity can be in fluid communication with the slot such that fluid and debris that enter the inner lumen at the second lateral passage can exit the inner lumen at the first lateral passage and into the lateral suction port. Accordingly, the need for secondary instrumentation to remove the debris can be reduced or eliminated.

The inner member can also have an alignment marker located at the distal end of the inner member. The alignment marker can be aligned with the first lateral passage where a position of the alignment marker correlates with a position of the first lateral passage. When the alignment marker is in a first position, this can correlate to the first lateral passage being in the first position and being aligned with the lateral suction portion. When the alignment marker is in a second position, this can correlate to the first lateral passage being the in second position and being misaligned with the lateral suction port.

Moreover, the inner member can include an electrode disposed at the inner member opening that extends between the inner member opening and a button tip. The electrode can include helical portions that can be used to ablate tissue at a target site. The helical portions of the electrode can be spaced apart from each other, such as to permit a fluid to travel between the spaced apart helical portions and into the inner member opening at the distal end of the inner member. The inner member opening can be in fluid communication with the inner lumen such that fluid that enters the inner member opening can exit the inner member via the inner lumen at the first lateral passage and into the suction port.

A potential advantage can include the ability to control suction by controlling a position of a first lateral passage of the hysteroscope with an alignment marker that can be viewable via a camera of an endoscope that uses the hysteroscope.

Another potential advantage can permit aspiration while providing or optimizing electrode efficiency.

A further potential advantage relates to allowing a user to maintain a clear field of view of a target site by enabling adjusting of an amount of suction occurring at the target site with the hysteroscope.

As another potential advantage, the present hysteroscope can obviate the need for additional instrumentation to remove debris from a target site since the debris can be pulled into the hysteroscope via a second lateral passage.

### BRIEF DESCRIPTION OF FIGURES

FIG. 1 illustrates an example of portions of a hysteroscope.

FIG. 2 shows an example of an inner member of the hysteroscope of FIG. 1.

FIG. 3 illustrates an example of a second lateral passage of the inner member of FIG. 1.

FIG. 4 illustrates an example of an electrode of the hysteroscope of FIG. 1.

FIG. 5 illustrates an example of a top view of an outer member of the hysteroscope of FIG. 1.

FIG. 6 shows an example of the inner member of FIG. 2 in a position different from the position shown in FIG. 2.

FIG. 7 illustrates another example of a top view of the outer member of the hysteroscope of FIG. 1.

FIGS. 8 and 9 show an example of the electrode of FIG. 4 in extended and retracted positions.

FIG. 10 illustrates an alternative example of a second lateral passage of the inner member of FIG. 1.

FIGS. 11 and 12 are alternative examples of electrodes that can be used with the hysteroscope of FIG. 1.

FIG. 13 is a flowchart indicating a reprocessing method for the hysteroscope of FIG. 1.

FIGS. 14 and 15 show an additional method of aligning a proximal passage aligns with a lateral suction port.

### DETAILED DESCRIPTION

A hysteroscope can have an outer member that defines an outer lumen along a longitudinal axis of the outer member. An inner member can be at least partially disposed within the outer lumen. The outer member can have a suction port that can be in fluid communication with the outer lumen. The inner member can be configured to rotate within the outer member. The inner member define an inner lumen along a longitudinal axis of the inner member. The inner member can also have a first lateral passage that can rotate into alignment with the suction port. Thus, fluid and debris within the inner member can exit into the suction port via the lateral passage. By the inner member being rotatable relative to the outer lumen, the inner member can rotate in order to adjust an alignment of the first lateral passage with the suction port such that the first lateral passage is at least partially aligned with the suction port.

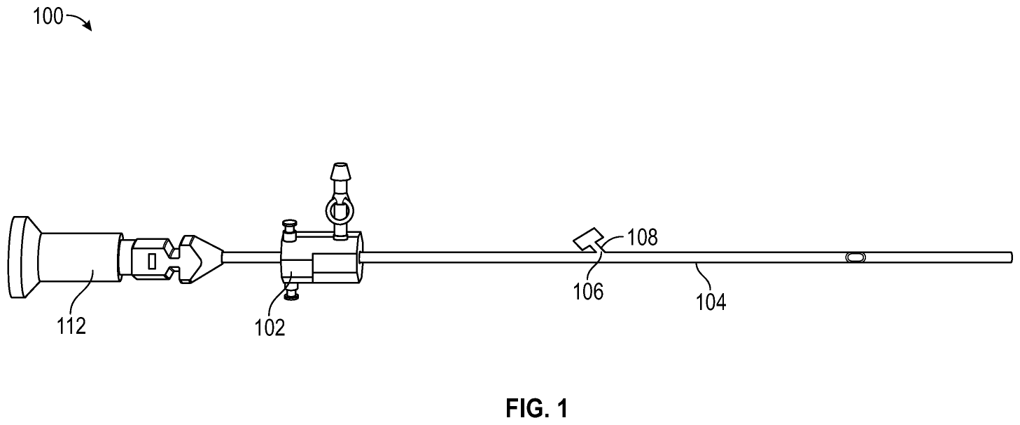

FIG. 1 shows an example of portions of a rigid or flexible hysteroscope 100. The hysteroscope 100 can include a housing 102, which can be configured to receive various components of the hysteroscope 100. Examples of such components can include, but are not limited to, an endoscope, a light source, an inflow channel, and an outflow channel.

An elongate outer member 104 can extend from the housing 102. The outer member 104 can include a lateral suction port 106 having a suction source connection 108. The suction source connection 108 can facilitate fluid coupling of the lateral suction port 106 with a suction source that can evacuate fluid from the lateral suction port 106. Thus, fluid and debris, such as material removed from a target site, can be removed from the target site and the hysteroscope 100 via the lateral suction port 106. In particular, fluid and debris can travel from the lateral suction port 106 along a suction port neck 108 to the suction source.

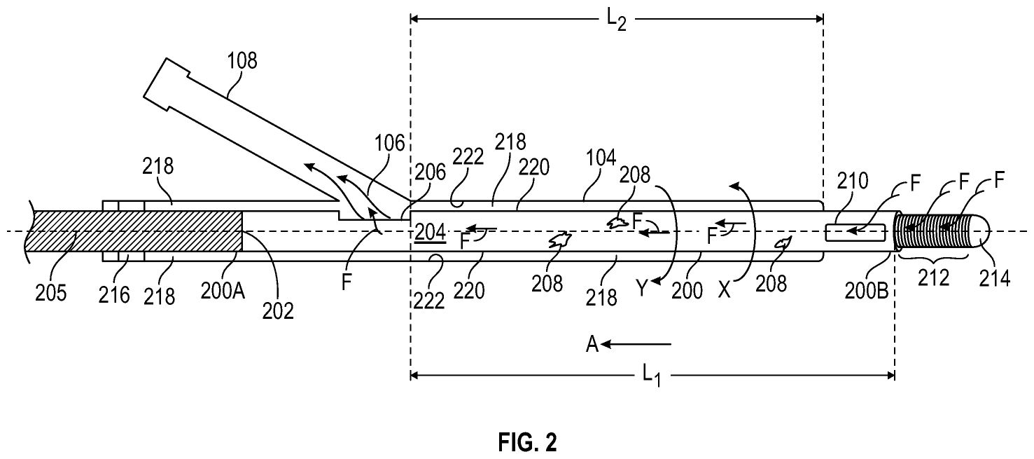

The hysteroscope 100 can also include a handle 112. The handle 112 can be used to control an elongate inner member 200, as shown in FIG. 2. The inner member 200 can be disposed within the outer member 104 and rotatable relative to the outer member 104 along a direction X or a direction Y. The inner member 200 can be closed at a proximal end 200A and open at a distal end 200B. The inner member 200 can include an obstruction 202, which can close off an inner lumen 204 defined within the inner member 200 and along a longitudinal axis 205. The obstruction 202 can be a sponge, seal, a stopper, a wall, or the like. The inner lumen 204 can be in fluid communication with the lateral suction port 106 via a proximal passage 206 in the inner member 200. The proximal passage 206 can be an opening in the inner lumen 204 that allows for the passage of fluid F and debris 208 that enters the inner lumen 204 to exit from the inner lumen 204 and into the lateral suction port 106.

The proximal passage 206 can be in different positions. In a first position, the proximal passage 206 aligns with the suction portion 106 such that the proximal passage 206 is at least partially aligned with the lateral suction port 106. In a second position, the proximal passage 206 can be partially aligned with the lateral suction port 106. By the inner lumen 204 being closed off by the obstruction 202, upon applying suction via the lateral suction port 106, the fluid F and the debris 208 within the inner lumen 204 can be pulled from the interior lumen 204 and into the lateral suction port 106.

The fluid F and the debris 208 can enter into the inner lumen 204 at the inner lumen distal end 200B. The inner member 200 can include a distal portion passage 210 that provides an opening to the inner lumen 204 through which the fluid F and the debris 208 can enter into the inner lumen 204 from a target site. The distal portion opening 210 can be in fluid communication with the inner lumen 204. Thus, as the fluid F and the debris 208 enter the distal portion opening 210 from a target site, the fluid F and the debris 208 can enter into the inner lumen 204, as shown in FIG. 2. The distal portion opening 210 can help reduce or eliminate the need to use a secondary instrument, such as a grasper or a basket, to remove the debris 208 from a target site.

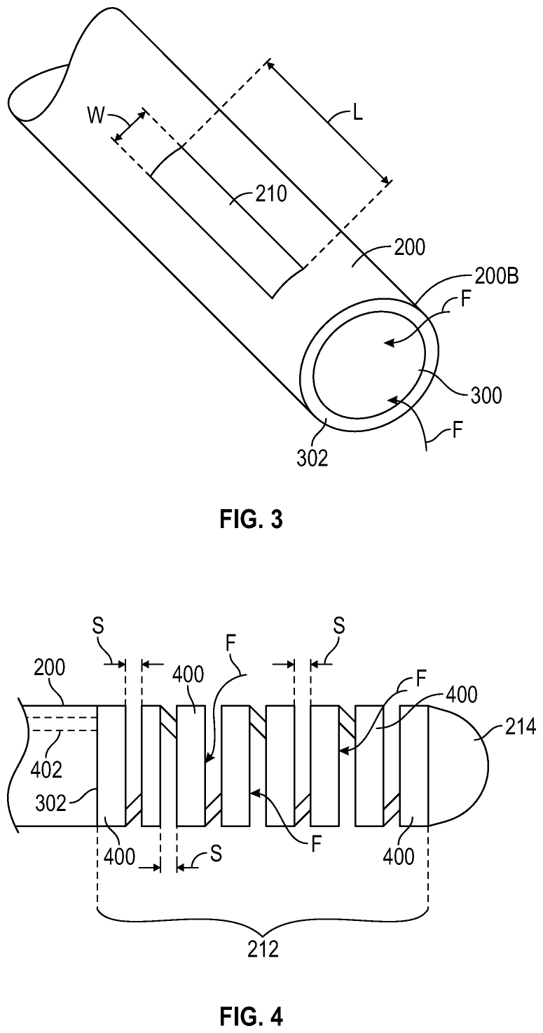

FIG. 3 shows the distal portion opening 210, which can have a length L in a range between about 2 millimeters to about 7 millimeters. Furthermore, the distal portion opening 210 can have a width W in a range between about 0.5 millimeters to about 2 millimeters. In addition to the distal portion opening 210, the inner member 200 can include an opening 300 at an inner member face 302 at the inner member end distal end 200B, as shown in FIG. 3. The distal end passage 300 can be located at the inner member distal end 200B. Besides the distal portion passage 210, the fluid F can enter the inner lumen 204 through the distal end passage 300. While the inner member 200 is shown as having both the distal portion opening 210 and the distal end passage 300, in alternative examples, the inner member can have only the distal portion opening 210 without the distal end passage 300. In addition, in alternative examples, the inner member can have only the distal end passage 300 without the distal portion passage 210. In addition, a combination of the distal portion opening 210 and the distal end passage 300 can be considered a single opening for the inner lumen 204. Moreover, a length L1 between the distal end passage 300 and the lateral suction port 106 is greater than a length L2 between the lateral suction port 106 and a distal end of the outer member 104, as shown with reference to FIG. 2.

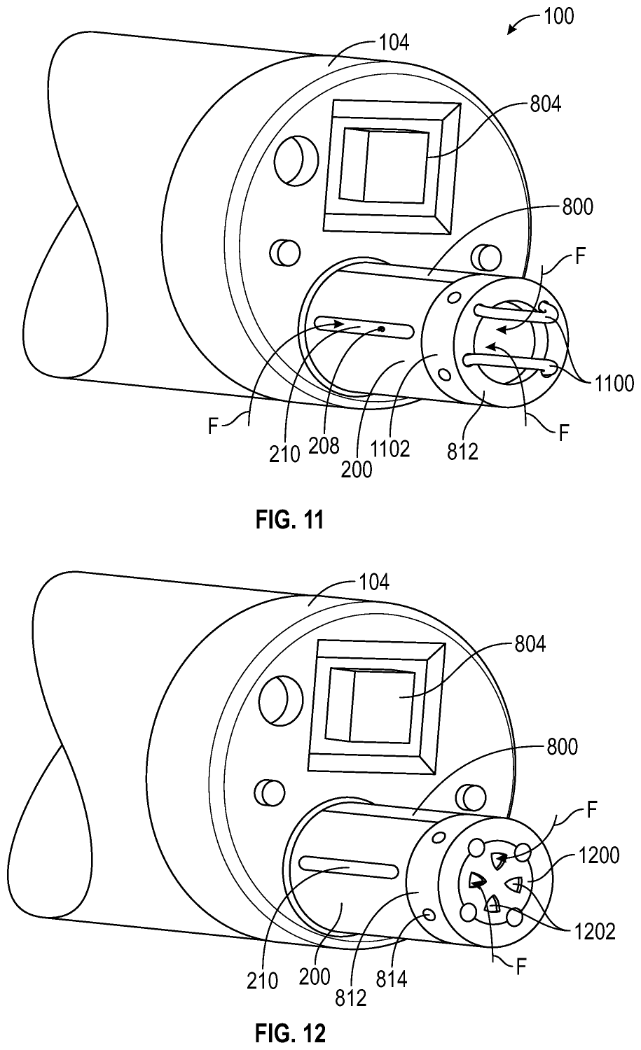

In FIG. 2, the hysteroscope 100 can also include an end effector 212 at the inner member distal end 200B. The end effector 212 can be an electrode and can extend between the inner member face 302 and a button tip 214. In addition to an electrode, the end effector can be any type of implement capable of removing tissue from a target site. In the example of FIG. 2, the end effector 212 can be a helical electrode. The end effector 212 can be used to resect tissue, such as polyps and fibroids, from a target site, such as a uterus. The end effector 212 can perform needle-like incisions of tissue or can be used for vaporizing tissue. During vaporization, the energy applied to end effector 212 can be controlled. The resulting energy applied by the end effector 212 to the target site can create a vapor pocket or a steam bubble. When the vapor pocket or steam bubble contacts tissue, such as a polyp or a fibroid, cellular rupture hemostasis can occur. During desiccation, bipolar energy from the end effector 212 can flow to tissue, such as a polyp or a fibroid, thereby dehydrating cells and causing hemostasis. While a helical electrode is shown, the end effector 212 can additionally or alternatively include one or more of a spring electrode, a bipolar loop electrode, a twizzle electrode, a wire grill electrode (FIG. 11), or any other implement capable of tissue resection.

In addition to the obstruction 202, the hysteroscope 100 can include a seal 216. The seal 216 can close off a working channel 218 formed between an outer surface 220 of the inner member 200 and an inner surface 222 of the outer member 104. The working channel 218 can be in fluid communication with the lateral suction port 106. Because the working channel 218 being closed off by the seal 216, pulling of the fluid F and the debris 208 from within the inner lumen 204 and into the lateral suction port 106 is further enhanced. The seal 216 can include any type of fluid tight seal between the inner member outer surface 220 and the outer member inner surface 222 that can allow rotation of the inner member 200 relative to the outer member 104 along the directions X and Y. Examples of the seal 216 can include a polymeric O-ring, a ceramic O-ring, a steel O-ring, or the like.

The fluid F can enter into the inner lumen 204 via the distal end passage 300 at the inner member distal end 200B. The fluid F can enter into the opening by traveling in between helical loops 400 of the electrode 212, such as shown in FIG. 4. One of the helical loops 400 can be coupled with the inner member face 302 while another of the helical loops 400 can be coupled with the button tip 214. One of the helical loops 400 can electrically couple with an electrical lead 402 disposed within the inner member 200. The electrical lead 402 can be embedded within the inner member 200. Alternatively, the inner member 200 can include a passageway within which the electrical lead 402 is located. The electrical lead 402 can be in electrical communication with a controlled power source and provide power to the helical loops 400.

The helical loops 400 can be separated from each other by a distance S. The space S can be in a range from about 0.25 millimeters to about 0.75 millimeters. The fluid F can travel through the space S and into the inner lumen 204 via the distal end passage 300. Once the fluid F travels into the inner lumen 204 via the distal end passage 300, the fluid F can exit the inner lumen 204 through the proximal passage 206 and the lateral suction port 106. The helical loops 400 can be formed from any type of conductive material, such as any type of steel, aluminum, copper, or the like.

The flow of the fluid F around the helical loops 400 can help provide a number of potential advantages. When the fluid F flows overs the helical loops 400 and into the distal end passage 300, the fluid F can cool the helical loops 400 during a procedure. Such cooling can help increase an efficiency of the electrode 212. This can result in more efficient tissue ablation thereby reducing the time necessary to perform a procedure with the hysteroscope 100. The flow of the fluid F around the helical loops 400 can also reduce an amount of bubbles that are created during use of the electrode 212, which can otherwise interfere with or obstruct a field of view.

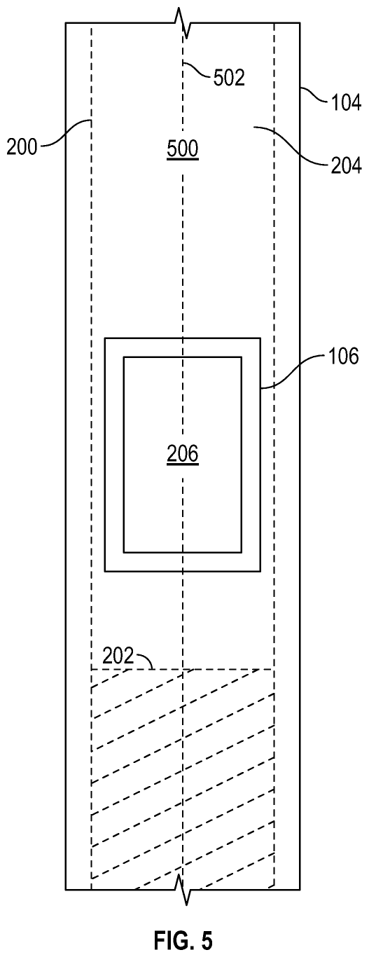

As noted above, the proximal passage 206 can have a number of positions that relate to an alignment of the proximal passage 206 with the lateral suction port 106. In a first position, as shown in FIGS. 2 and 5, the proximal passage 206 can be aligned with the lateral suction port 106. For ease of reference, the suction port connection 108 is not shown. In the first position, when the proximal passage 206 is aligned with the lateral suction port 106, the proximal passage 206 is aligned with the lateral suction port 106 such that no portion of the slot 106 is obstructed. Thus, the proximal passage 206 is at least partially aligned with the lateral suction port 106.

In FIG. 5, the inner member 200 can reside within a lumen 500 defined by the outer member 104 and in fluid communication with the lateral suction port 106. The lumen 500, along with the outer member 104, can define a longitudinal axis 502. The inner member 200 can be at least partially disposed within the lumen 500.

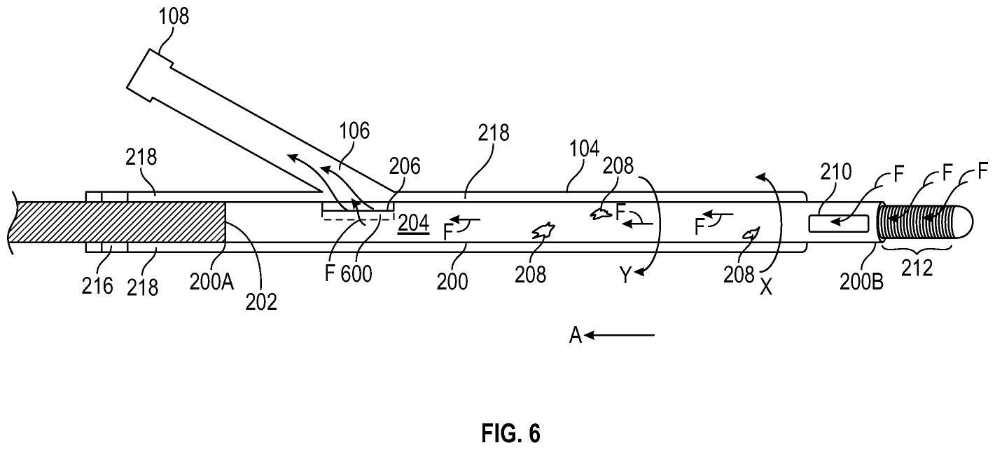

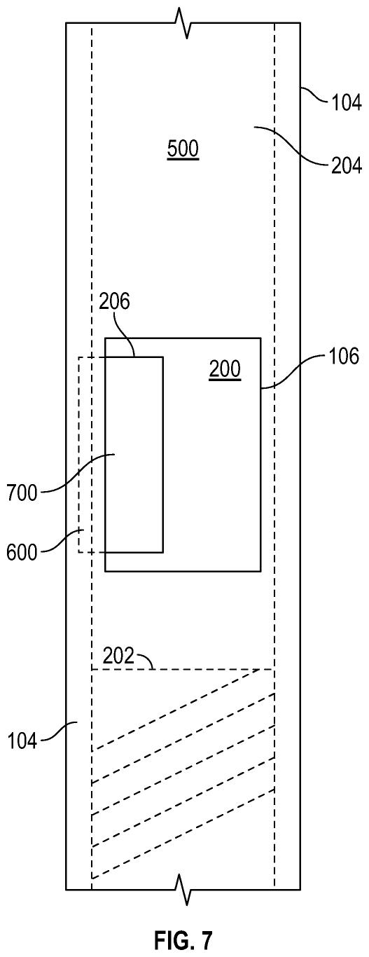

In a second position, as shown in FIGS. 6 and 7, the proximal passage 206 is misaligned with the lateral suction port 106. More specifically, a first portion 600 of the proximal passage 206 is obstructed by the outer member 104. Thus, only a second portion 700 of the first lateral passage is unobstructed relative to the lateral suction port 106. While the proximal passage 206 is shown as being smaller than the lateral suction port 106, the proximal passage 206 can be the same size as the lateral suction port 106 or it can be larger than the lateral suction port 106. In addition, while two positions of the proximal passage 206 are shown in FIGS. 5-7, the proximal passage 206 can have any number of positions. To further illustrate, the proximal passage 206 can be positioned such that no portion of the proximal passage 206 is aligned with the lateral suction port 106.

The inner member 200 can be rotated along the direction X or the direction Y in order to move the proximal passage 206 into different positions relative to the lateral suction port 106, such as the first and second positions. As noted above, the inner member 200 can be coupled with the handle 112. A user of the hysteroscope 100 can rotate the handle 112, thereby rotating the inner member 200 along the direction X or the direction Y while the outer member 104 remains stationary. When the inner member 200 rotates, the proximal passage 206 can be placed into the first position where the proximal passage 206 aligns with the lateral suction port 106. Furthermore, when the inner member 200 rotates, the proximal passage 206 can also be rotated such that the proximal passage 206 moves into the second position where the proximal passage 206 becomes misaligned with the lateral suction port 106. The degree of suction passed from the lateral suction port 106 to the proximal passage 206 can be controlled, such as by controlling a degree of misalignment therebetween.

To help with moving the proximal passage 206 into the first position, the hysteroscope 100 can include an alignment marker 800 located at the distal end 200B of the inner member 200, as shown with reference to FIG. 8. The alignment marker 800 can be alignable with the proximal passage 206 and can correspond to a position of the proximal passage 206. When the alignment marker 800 has the position shown in FIG. 8, the proximal passage 206 can be in the first position where the proximal passage 206 aligns with the lateral suction port 106. Moreover, when the alignment marker 800 is in the position shown with dotted lines 802, the proximal passage 206 can be misaligned with the suction port 103.

The hysteroscope 100 can include a camera 804 having a field of view 806 at a distal end of the outer member 104. Using the camera 804, a user of the hysteroscope 100 can see when a position of the alignment marker 800 is within the field of view 806. This can help the user determine a position of the proximal passage 206 relative to the lateral suction port 106, e.g., whether or not the proximal passage 206 is in one of the first or second positions described herein or any other position. By positioning the alignment marker 800 as desired within the field of view 806, a user can control the amount of suction at a target site using the alignment marker 800. Thus, if the field of view 806 becomes cloudy, the user can reduce the amount of suction by rotating the handle 112, which ultimately rotates the inner member 200 and the proximal passage 206 to cause or increase misalignment of the proximal passage 206 relative to the lateral suction port 106. When misalignment occurs or increases, a suction rate can decrease. Thus, irrigant provided to the target site from outflow ports 808 of the hysteroscope 100 can be used to clear the field of view 806.

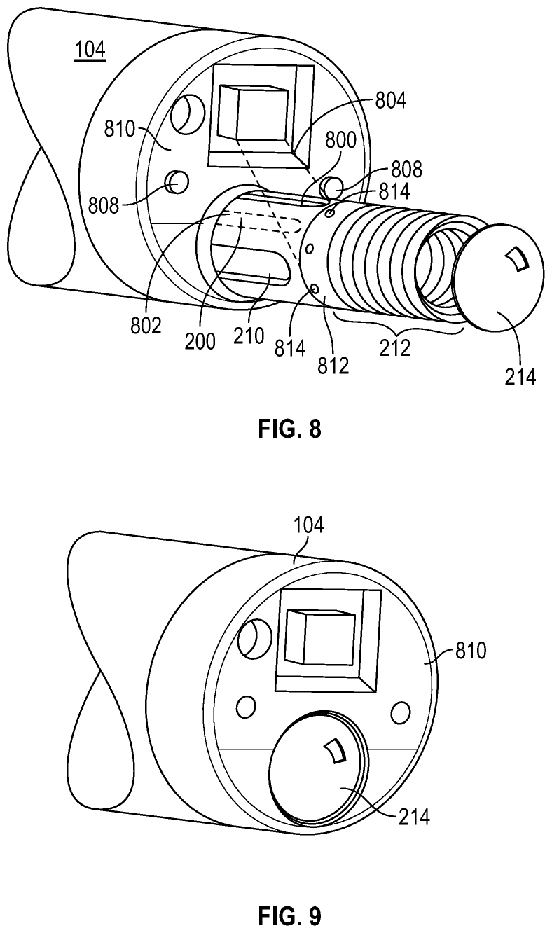

In FIG. 8, the hysteroscope 100 is shown in an extended position. In this extended position, the inner member 200 extends out of a face 810 of the outer member 104. The inner member 200 can be slidable relative to the outer member 104 between the extended position and a retracted position. When a user is positioning the hysteroscope 100 in a patient, such as moving the hysteroscope 100 through a vaginal canal and a cervix of the patient, the hysteroscope 100 can have a retracted position. In this retracted position, the inner member 200 is retracted into the hysteroscope 100, as shown in FIG. 9. In the retracted position, the inner member 200 is retracted into the outer member 104 such that the button tip 214 is flush with the outer lumen face 810. As such, the possibility of the inner member 200 coming into contact with the patient during positioning of the hysteroscope 100 within the patient is reduced or minimized. This can help minimize the possibility of the inner member 200 causing trauma to the patient.

The inner member 200 can be formed from a metal, such as any type of steel, aluminum, or the like. The inner member 200 can include a ceramic insulator 812. The ceramic insulator 812 can be in the shape of a collar. The ceramic insulator 812 can help separate and electrically isolate the end effector 212 from the inner member 200. The ceramic insulator 812 can be located at the inner lumen distal end 200B. The ceramic insulator 812 can couple with the distal end passage 300, thereby coupling with the inner member 200. The ceramic insulator 812 can have inflow ports 814 in fluid communication with the inner lumen 204. Fluid F can travel through the ceramic insulator inflow ports 814 during a hysteroscopic procedure and into the inner lumen 204. This can help increase and improve the suction capability of the hysteroscope 100. In addition, the ceramic insulator 812 can define an opening (FIG. 11) that can be in fluid communication with the distal end passage 300.

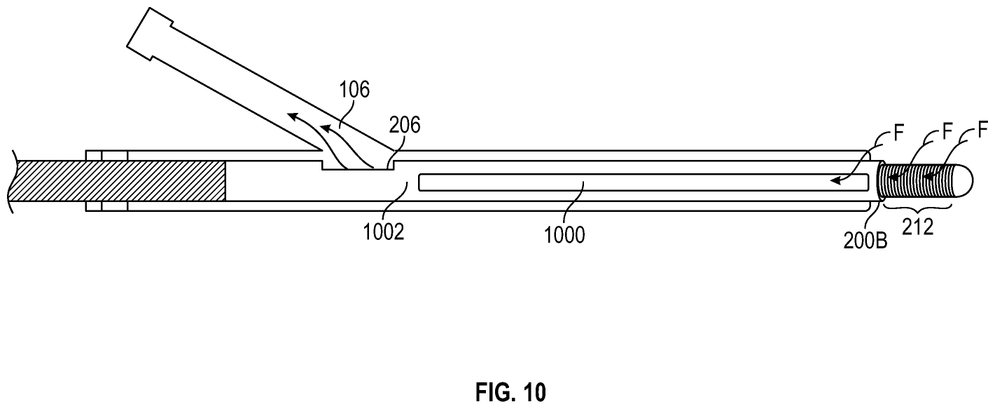

The proximal passage 206 can be located at the inner lumen distal end 200B. Alternatively, the hysteroscope 100 can include a lateral passage 1000 that can extend from the inner lumen distal end 200B to a terminus 1002, as shown in FIG. 10. The lateral passage 1000 can continuously extend from the inner member distal end 200B to the lateral suction port 106 and the proximal passage 206 at the terminus 1002. The lateral passage 1000 can be larger than the distal portion passage 210.

In FIGS. 1-10, the end effector 212 is shown with a helical configuration. But the hysteroscope 100 can include electrodes having different configurations. FIG. 11 shows an example in which the inner member 200 can include a grill formed from grill components 1100. The grill can be electrically conductive, such as to serve as an electrode for the hysteroscope 100. The ceramic insulator 812 can define an opening 1102 that can be in fluid communication with the distal end passage 300. The grill components 1100 can span across the ceramic insulator opening 1102 as shown in FIG. 11 such that the grill formed by the grill components 1100 can span across the ceramic insulator opening 1102. The grill components 1100 can be formed from any type of conductive material, such as any type of steel, aluminum, copper, or the like.

FIG. 11 shows the inner member 200, which can retract into the outer member 104 and can extend from the outer member 104 at a target site. The fluid F can travel over the grill components 1100, similar to that described above with reference to the helical loops 400. This can provide similar benefits to the grill components 1100 and the grill formed by the grill components 1100 as detailed above with reference to the electrode 212. The hysteroscope 100 shown in FIG. 11 can also include the proximal passage 206 (shown) or the lateral passage 1000 along with the alignment marker 800 and the camera 804.

In addition to the end effector 212 and the grill defined by the grill components 1100, the hysteroscope 100 can also have an electrode 1200 that includes inflow ports 1202, as shown in FIG. 12. The electrode 1200 can be located on the ceramic insulator 812. The inflow ports can permit fluid inflow through the electrode 1200 and into the inner lumen 204. The electrode 1200 can be formed from any type of conductive material, such as any type of steel, aluminum, copper, or the like. The electrode 1200 can be used to resect tissue from a target site during use of the hysteroscope 100. The inner member 200 of FIG. 12 can retract into the outer member 104 as detailed above and extend from the outer member 104 at a target site also as discussed above. The fluid F can travel over the electrode 1200 and into the electrode inflow ports 1202. The passage of the fluid F over the electrode 1200 can provide the benefits to the electrode 1200 as detailed above with reference to the electrode 212. The hysteroscope 100 shown in FIG. 12 can also include the proximal passage 206 (shown) or the lateral passage 1000 along with the alignment marker 800 and the camera 804.

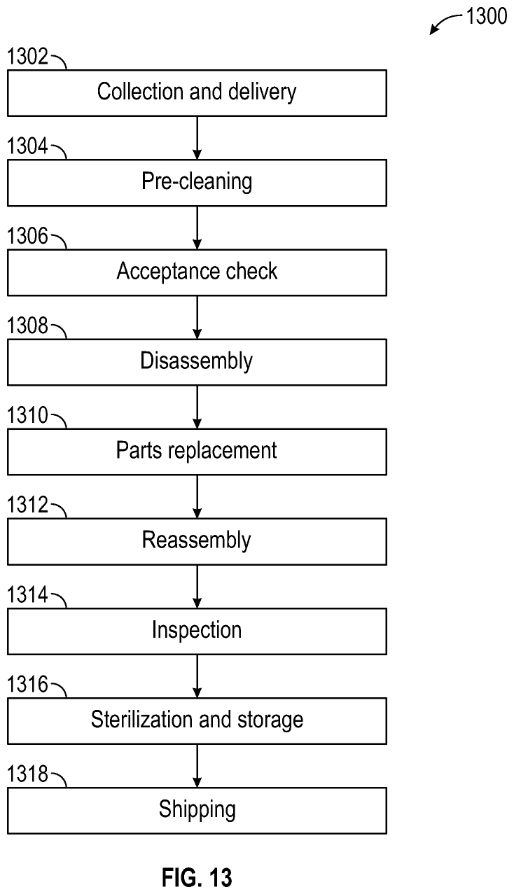

The hysteroscope 100 can be disposed of after a single use. Alternatively, the hysteroscope 100 can be repeatedly used a plurality of times. When the hysteroscope 100 can be repeatedly used a plurality of times, the hysteroscope 100 can be subjected to a method 1300, as shown in FIG. 13, which is a flowchart illustrating a reprocessing method for the hysteroscope 100. The hysteroscope 100 described above may be disposed of after one use or may be repeatedly used a plurality of times. When repeatedly used a plurality of times, the reprocessing method in FIG. 13 can be used. An operator who remanufactures devices can collect the used hysteroscope 100 after it has been used for treatment and can transport the hysteroscope 100 to a factory or other facility at 1302. The used hysteroscope 100 can be transported in a dedicated container such as to help prevent contamination from other treatments.

At 1304, the operator cleans and sterilizes the used hysteroscope 100. During cleaning, deposits adhering to the portions of the hysteroscope 100 are removed by using a brush or the like. Any cleaning solution of isopropanol-containing cleaning agent, proteolytic enzyme detergent, and alcohol can be applied to the hysteroscope 100 in order to remove pathogenic microorganisms and the like derived from blood, body fluid, or the like. The cleaning agent is not limited to the cleaning liquid described above, and other cleaning agents can be used. During sterilization, high-pressure steam sterilization, ethylene oxide gas sterilization, gamma ray sterilization, hydrogen peroxide and hydrogen peroxide low temperature sterilization can be applied to the hysteroscope 100. By virtue of the hysteroscope 100 having the structures described above, the hysteroscope 100 is easy to clean.

After cleaning and sterilization, an acceptance check of the hysteroscope 100 can be performed at 1306. During the acceptance check, an inspection of the hysteroscope 100 can be performed to determine if the hysteroscope 100 has any significant defects. Moreover, a number of times the hysteroscope 100 has been reprocessed can be determined. The number of times the hysteroscope 100 has been reprocessed can be compared against a threshold to determine if the number of times the hysteroscope 100 has been reprocessed exceeds a threshold and should no longer be used.

Next, at 1308, the hysteroscope 100 can be disassembled where various components of the hysteroscope 100, such as the outer member 104, the inner member 200, the electrodes 212 and 1200, or grill components 1100 are removed from the hysteroscope 100. While only the outer member 104, the inner member 200, the electrodes 212 and 1200, and grill components 1100 are referenced as being removed, any portion of the hysteroscope 100 described herein can be removed at 1308.

After disassembly at 1308, any components of the hysteroscope 100 that are deemed defective can be replaced at 1310. To further illustrate, if the end effector 212 is deemed defective, the end effector 212 can be replaced at 1310. While the end effector 212 is mentioned as being replaced, any portion of the hysteroscope 100 described herein can be replaced at 1310.

After components of the hysteroscope 100 are replaced at 1310, the hysteroscope 100 can be reassembled at 1312. During reassembly, an identifier that can indicate the hysteroscope 100 has been modified from its original condition to include the replacement component(s) can be added. The identifier can include a label or any other type of indicia that designates the hysteroscope 100 as reprocessed, refurbished, or remanufactured.

The hysteroscope 100 is then inspected and tested at 1314. Specifically, a user can verify that the newly formed hysteroscope 100 has the same effectiveness and safety as the original product by various functional tests. After inspection, the hysteroscope 100 can be sterilized and stored at 1316. The hysteroscope 100 can be sterilized with a sterilizing gas such as ethylene oxide gas or propylene oxide gas. After sterilization, the hysteroscope 100 can be stored at 1316 and then subsequently shipped at 1318.

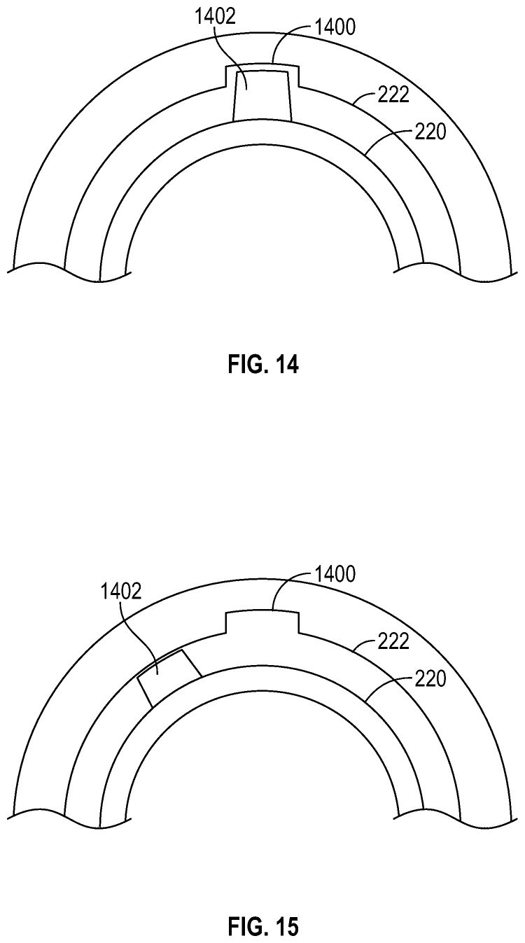

The alignment marker 800 was described above as being used to align proximal passage 206 aligns with the lateral suction port 106. In further examples, the outer member inner surface 222 can include a groove 1400 and the inner member outer surface 220 can include a projection 1402 as shown in FIG. 14. The projection 1402 can rest within the groove 1400 when the proximal passage 206 aligns with the lateral suction port 106. The projection 1402 can be spring loaded such that the projection 1402 moves into the groove 1400 upon alignment with the groove 1400. Moreover, when the projection 1402 is not disposed within groove 1400, as shown in FIG. 15, proximal passage 206 may be in misalignment with the lateral suction port 106.

Having described various aspects and features of the present subject matter, the following numbered examples are provided as illustrative embodiments:

Example 1 is a medical device comprising: an elongate inner member disposed at least partially within an elongate outer member defining an outer first lumen along a longitudinal axis of the outer member and a lateral suction port at the elongate outer member, the lateral suction port in fluid communication with the outer lumen, wherein the inner member defines a inner lumen along a longitudinal axis of the inner member, the inner member comprising wherein: the inner member comprises a first passage on a distal end portion of the inner member in fluid communication with the inner lumen and a second passage a first lateral passage an opening in the inner member, the opening first lateral passage rotatable disposable configurable to be in into at least partial alignment with the a lateral suction port of an outer member lumen to provide fluid communication between the lateral suction port and the second inner lumen; and an end effector at a distal end of the inner member.

In Example 2, the subject matter of Example 1 includes, wherein the first passage comprising a front opening at a distal end of the inner member and a side opening on a side wall of the inner member at the distal end portion in fluid communication with the second lumen.

In Example 3, the subject matter of Examples 1-2 includes, an elongate outer member; and a camera at a distal end of the elongate outer member.

In Example 4, the subject matter of Example 3 includes, an alignment marker located at the distal end the inner member, the alignment marker being alignable with the second passage and positionable in a field of view of the camera.

In Example 5, the subject matter of Examples 3-4 includes, wherein the inner member is slidable relative to the outer member between a retracted position and an extended position.

In Example 6, the subject matter of Examples 3-5 includes, an alignment marker located at the distal end of the inner member, the alignment marker being alignable with the second passage and positionable in a field of view of the camera.

In Example 7, the subject matter of Examples 3-6 includes, a working channel located between an inner surface of the outer member and an outer surface of the inner member, the working channel being in fluid communication with the lateral suction port.

In Example 8, the subject matter of Examples 3-7 includes, wherein the second passage is disposed more proximal than the first passage, and a length between the first passage and the second passage is more than a length between a distal end of the outer member and the lateral suction port.

In Example 9, the subject matter of Examples 1-8 includes, wherein the end effector is a helical electrode defining helical loops adjacently spaced apart from each other to allow fluid flow therebetween.

In Example 10, the subject matter of Examples 1-9 includes, a ceramic insulator coupled to the inner member to separate the inner member from the end effector.

In Example 11, the subject matter of Examples 9-10 includes, wherein the end effector is located on the ceramic insulator and the end effector includes fluid inflow ports permitting fluid inflow through the end effector and into the inner lumen defined by the inner member and wherein the ceramic insulator defines a ceramic insulator opening in fluid communication with the inner lumen in the inner member.

In Example 12, the subject matter of Example 11 includes, wherein the electrode includes a grill spanning across the ceramic insulator opening.

In Example 13, the subject matter of Examples 11-12 includes, wherein the ceramic insulator includes an inflow port in fluid communication with the inner lumen defined by the inner member.

In Example 14, the subject matter of Examples 1-13 includes, a working channel located between an inner surface of the outer member of and an outer surface of the inner member, the working channel being in fluid communication with the lateral suction port.

In Example 15, the subject matter of Examples 1-14 includes, wherein the end effector is a helical electrode defining helical loops adjacently spaced apart from each other to allow fluid flow therebetween.

In Example 16, the subject matter of Examples 1-15 includes, a seal disposed more proximal than the second passage.

In Example 17, the subject matter of Examples 1-16 includes, a seal disposed between an outer surface of the inner member and an inner surface of the outer member and more proximal than the second passage.

Example 18 is a method for processing an instrument for surgery, the method comprising: sterilizing the medical device of Example 1; and storing the medical device in a sterile container.

Example 19 is a medical device comprising: an elongate inner member disposed at least partially within an elongate outer member defining an outer lumen along a longitudinal axis of the outer member and a lateral suction port at the elongate outer member, the lateral suction port in fluid communication with the outer lumen, wherein the inner member defines a inner lumen along a longitudinal axis of the inner member, wherein: the inner member comprises a first passage on a distal end portion of the inner member in fluid communication with the inner lumen and a second passage configurable to be in at least partial alignment with the lateral suction port of the outer member to provide fluid communication between the lateral suction port and the inner lumen; and an elongate inner member disposed at least partially within an elongate outer member defining a first lumen along a longitudinal axis of the outer member and a lateral suction port at the elongate outer member, the lateral suction port in fluid communication with the first lumen, wherein the inner member defines a second lumen along a longitudinal axis of the inner member, the inner member comprising: an opening in the inner member, the opening configurable to be in at least partial alignment with a lateral suction port of an outer lumen to provide fluid communication between the lateral suction port and the second lumen; an end effector at a distal end of the inner member; and a working channel disposed between an inner surface of the outer lumen and an outer surface of the inner lumen, the working channel being in fluid communication with the suction port.

Example 20 is a method for processing an instrument for surgery, the method comprising: sterilizing the medical device of Example 19; and storing the medical device in a sterile container.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

What is claimed is:

1. A medical device comprising:
an elongate inner member disposed at least partially within an elongate outer member defining an outer lumen along a longitudinal axis of the outer member and a lateral suction port at the elongate outer member, the lateral suction port in fluid communication with the outer lumen, wherein the inner member defines an inner lumen along a longitudinal axis of the inner member, wherein:
the inner member comprises a first passage on a distal end portion of the inner member in fluid communication with the inner lumen and a second passage configurable to be in at least partial alignment with the lateral suction port of the outer member to provide fluid communication between the lateral suction port and the inner lumen; and
an end effector at a distal end of the inner member.

2. The medical device of claim 1, wherein the first passage comprising a front opening at a distal end of the inner member and a side opening on a side wall of the inner member at the distal end portion in fluid communication with the second lumen.

3. The medical device of claim 1, further comprising:
an elongate outer member; and
a camera at a distal end of the elongate outer member.

4. The medical device of claim 3, further comprising an alignment marker located at the distal end the inner member, the alignment marker being alignable with the second passage and positionable in a field of view of the camera.

5. The medical device of claim 3, wherein the inner member is slidable relative to the outer member between a retracted position and an extended position.

6. The medical device of claim 3, further comprising an alignment marker located at the distal end of the inner member, the alignment marker being alignable with the second passage and positionable in a field of view of the camera.

7. The medical device of claim 3, further comprising a working channel located between an inner surface of the outer member and an outer surface of the inner member, the working channel being in fluid communication with the lateral suction port.

8. The medical device of claim 3, wherein the second passage is disposed more proximal than the first passage, and
a length between the first passage and the second passage is more than a length between a distal end of the outer member and the lateral suction port.

9. The medical device of claim 1, wherein the end effector is a helical electrode defining helical loops adjacently spaced apart from each other to allow fluid flow therebetween.

10. The medical device of claim 1, further comprising a ceramic insulator coupled to the inner member to separate the inner member from the end effector.

11. The medical device of claim 9, wherein the end effector is located on the ceramic insulator and the end effector includes fluid inflow ports permitting fluid inflow through the end effector and into the inner lumen defined by the inner member and wherein the ceramic insulator defines a ceramic insulator opening in fluid communication with the inner lumen in the inner member.

12. The medical device of claim 11, wherein the electrode includes a grill spanning across the ceramic insulator opening.

13. The medical device of claim 11, wherein the ceramic insulator includes an inflow port in fluid communication with the inner lumen defined by the inner member.

14. The medical device of claim 1, further comprising a working channel located between an inner surface of the outer member of and an outer surface of the inner member, the working channel being in fluid communication with the lateral suction port.

15. The medical device of claim 1, wherein the end effector is a helical electrode defining helical loops adjacently spaced apart from each other to allow fluid flow therebetween.

16. The medical device of claim 1, further comprising a seal disposed more proximal than the second passage.

17. The medical device of claim 1, further comprising a seal disposed between an outer surface of the inner member and an inner surface of the outer member and more proximal than the second passage.

18. A method for processing an instrument for surgery, the method comprising:
sterilizing the medical device of claim 1; and
storing the medical device in a sterile container.

19. A medical device comprising:
an elongate inner member disposed at least partially within an elongate outer member defining an outer lumen along a longitudinal axis of the outer member and a lateral suction port at the elongate outer member, the lateral suction port in fluid communication with the outer lumen, wherein the inner member defines a inner lumen along a longitudinal axis of the inner member, wherein:
the inner member comprises a first passage on a distal end portion of the inner member in fluid communication with the inner lumen and a second passage configurable to be in at least partial alignment with the lateral suction port of the outer member to provide fluid communication between the lateral suction port and the inner lumen; and
a working channel disposed between an inner surface of the outer lumen and an outer surface of the inner lumen, the working channel being in fluid communication with the suction port.

20. A method for processing an instrument for surgery, the method comprising:
sterilizing the medical device of claim 19; and
storing the medical device in a sterile container.
